(19) United States
(12) Patent Application Publication
(10) Pub. No.: US 2016/0109355 A1
(43) Pub. Date: Apr. 21, 2016

(54) DATA ANALYSIS APPARATUS AND DATA ANALYSIS METHOD

(71) Applicant: toor Inc., Onuma-gun (JP)

(72) Inventors: Yoshio TAKAEDA, Onuma-gun (JP); Tetsuya KANADA, Onuma-gun (JP)

(21) Appl. No.: 14/871,606

(22) Filed: Sep. 30, 2015

(30) Foreign Application Priority Data
Oct. 15, 2014 (JP) 2014-210671
Jun. 8, 2015 (JP) 2015-1155600
Sep. 7, 2015 (JP) 2015-175833

(51) Int. Cl. G01N 19/08; A61B 5/11; A61B 5/0402; G01B 5/28; A61B 5/08

(52) U.S. Cl. CPC G01N 19/08; G01B 5/28; A61B 5/08; A61B 5/0402; A61B 5/11

(57) ABSTRACT

The data analysis apparatus according to the invention includes an indexing unit configured to calculate indexes indicating similarity between a plurality of segments, each of which is numerical data having at least one frequency component, using a frequency spectrum containing the frequency component of the segment, and a map creating unit configured to plot the segment on a map based on indexes.

### TECHNICAL FIELD

The present invention relates to a data analysis apparatus and a data analysis method through which numerical data containing frequency components is analyzed.

### BACKGROUND ART

Vibration analysis methods of detecting an abnormality of a path for transportation, such as a railroad or a road are suggested (for example, see Patent Literatures 1 and 2). In the method of Patent Literature 1, a transfer function at a normal time is acquired in advance, And the abnormality is detected based on the transfer function. In the method of Patent Literature 2, a reference frequency spectrum at a normal time is acquired in advance, and the abnormality is detected based on the reference frequency spectrum.

The transfer function and the reference frequency spectrum at a normal time vary according to the characteristic of the path. Therefore, in the vibration analysis methods of Patent Literatures 1 and 2, there is a need to measure the transfer functions or the reference frequency spectrums at a normal time with respect to all the paths where the abnormality may be detected.

### CITATION LIST

### Patent Literature

Patent Literature 1: JP 2992954 B1
Patent Literature 2: JP 5130181 B1

### SUMMARY OF INVENTION

### Technical Problem

The path for transportation, such as the railroad and the road is always repaired, so that information at a normal time as a reference, such as the transfer function and the reference frequency spectrum, is always changed. Therefore, it is difficult to hold the information at a normal time as a reference with respect to all the paths where the abnormality may be detected. Furthermore, even when a deviation from the reference spectrum can be detected, another configuration is necessary for determining a degradation state and it is difficult to determine the degradation state at the same time.

An object of the invention is to provide a data analysis apparatus and a data analysis method of numerical data containing a frequency component, which can diagnose the abnormality and a state of the abnormality without using information at a normal time as a reference.

### Solution to Problem

A data analysis apparatus according to the present invention includes:

an indexing unit configured to calculate indexes each indicating similarity between each pair in a plurality of segments, each of which is numerical data having at least one frequency component, using a frequency spectrum containing the frequency component of the segment; and a map creating unit configured to plot the segments on a plane based on the indexes.

The data analysis apparatus according to the present invention may further include an area selecting unit configured to select a certain area in which the segments are plotted on the plane.

The data analysis apparatus according to the present invention may further include a characteristic spectrum extracting unit configured to extract a characteristic frequency or the frequency spectrum of all segments in the area selected by the area selecting unit.

In the data analysis apparatus according to the present invention, the numerical data contains vibration frequency components of a vehicle, and wherein the certain area corresponds to a degradation or a state of the vehicle or a path where the vehicle runs, or a driving state of the vehicle.

In the data analysis apparatus according to the present invention, the numerical data includes vibration frequency components of which at least a part of a biological body, and

wherein the certain area corresponds to a state of the part or all of the biological body.

In the data analysis apparatus according to the present invention, the indexing unit calculates new indexes indicating similarity between a new segment different from the plurality of segments and the plurality of segments using the frequency spectrum of the new segments and spectrums of the plurality of segments,

the data analysis apparatus further comprising a plotting unit configured to plot the new segment in the vicinity of a segment similar to the new segment among the plurality of segments based on the new indexes.

The data analysis apparatus according to the present invention may further include a division unit configured to divide the numerical data into spatial or temporal sections to create the segments.

According to the present invention, a data analysis method which is performed by a data analysis apparatus, includes:

an indexing procedure in which the data analysis apparatus calculates indexes indicating similarity between a plurality of segments, each of which is numerical data having at least one frequency component, using a frequency spectrum containing the frequency component of the segment; and

a map creating procedure in which the data analysis apparatus plots the segments on a plane based on the indexes.

Further, the above-described inventions can be combined to each other to the extent possible.

### Advantageous Effects of Invention

According to the invention, since the indexes indicating similarity between each pair of segments is used, it is possible to detect an abnormal segment to determine an abnormal state without using information at a normal time.

### BRIEF DESCRIPTION OF DRAWINGS

FIG. 1 is a diagram illustrating an example of a vibration analysis system according a first embodiment;

FIG. 2 is a diagram illustrating an example of a map;

FIG. 3 is a diagram illustrating an example of a vibration analysis system according to a fifth embodiment;

FIG. 4 is a diagram illustrating an example of a map in a sixth embodiment; and

FIG. 5 is a diagram illustrating an example of a segment in the sixth embodiment.

### DESCRIPTION OF EMBODIMENTS

Hereinafter, embodiments of the invention will be described in detail with reference to the drawings. Further, the invention is not limited to the following embodiments. These embodiments are given as merely exemplary, and the invention can be variously changed and improved based on knowledge of a person skilled in the art. Further, the components denoted with the same symbols in the specification and the drawings indicate the same components.

### First Embodiment

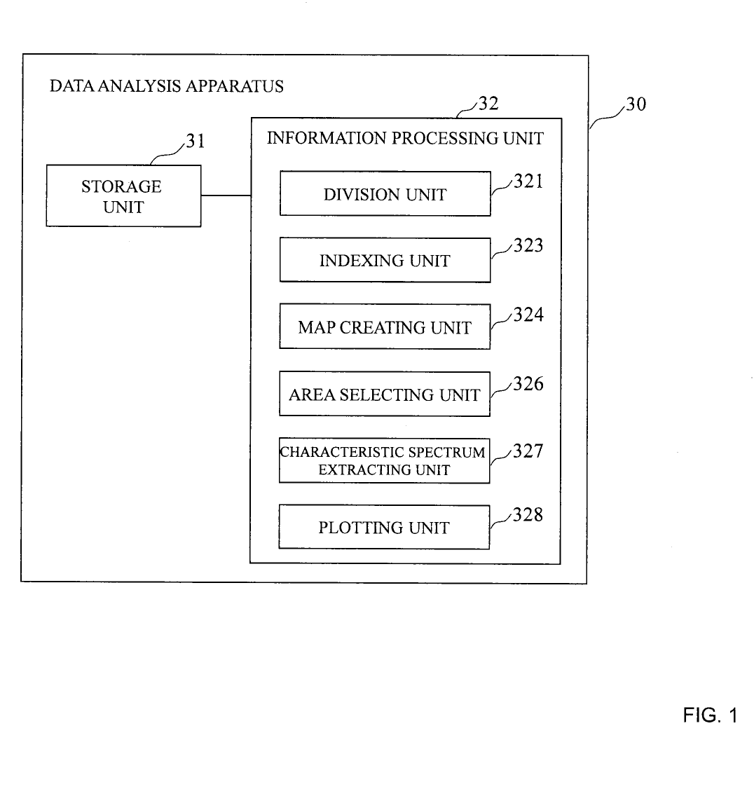

FIG. 1 illustrates an example of a data analysis apparatus according to this embodiment. A data analysis apparatus 30 according to this embodiment includes a storage unit 31 and an information processing unit 32.

The storage unit 31 stores various types of numerical data therein. Numerical data stored in the storage unit 31 has at least one frequency component and a numerical value indicating an amplitude of that frequency. The numerical data is a quantifiable “amount”, and any data may be employed as long as the data can be converted into a frequency component (for example, time or a spatial frequency).

In addition, the numerical data stored in the storage unit 31 is stored in a state where the data is divided into a plurality of segments. The segment is a part of the original numerical data which is divided based on arbitrary attribute data other than the frequency among the attribute data contained in the numerical data. Examples of such attribute data may include a date, a position, and an individual ID. A set of the original segments is called a “segment parent population”. Frequency spectrum data (hereinafter, referred to as “segment data”) for each segment is obtained by converting the original data of the segment into the temporal or the spatial frequency.

The information processing unit 32 reads the numerical data out of the storage unit 31 and performs a data analysis method according to this embodiment. In the data analysis method according to this embodiment, the information processing unit 32 sequentially performs an indexing procedure and a map creating procedure.

In the indexing procedure, an indexing unit 323 reads the numerical data out of the storage unit 31, and calculates indexes indicating similarity between the plurality of segments using the frequency spectrum containing the frequency component of the segment.

Regarding the frequency and its amplitude of the segment data spectrum as the dimension and its value respectively, the segment data can be denoted as a vector in a multi-dimensional space having an extensive number of dimensions. Similarity between two segment data is represented by a distance between the corresponding vectors. In general, an inner product or an outer product may be employed besides a direct distance as the mutual distance between the vectors in a vector space model.

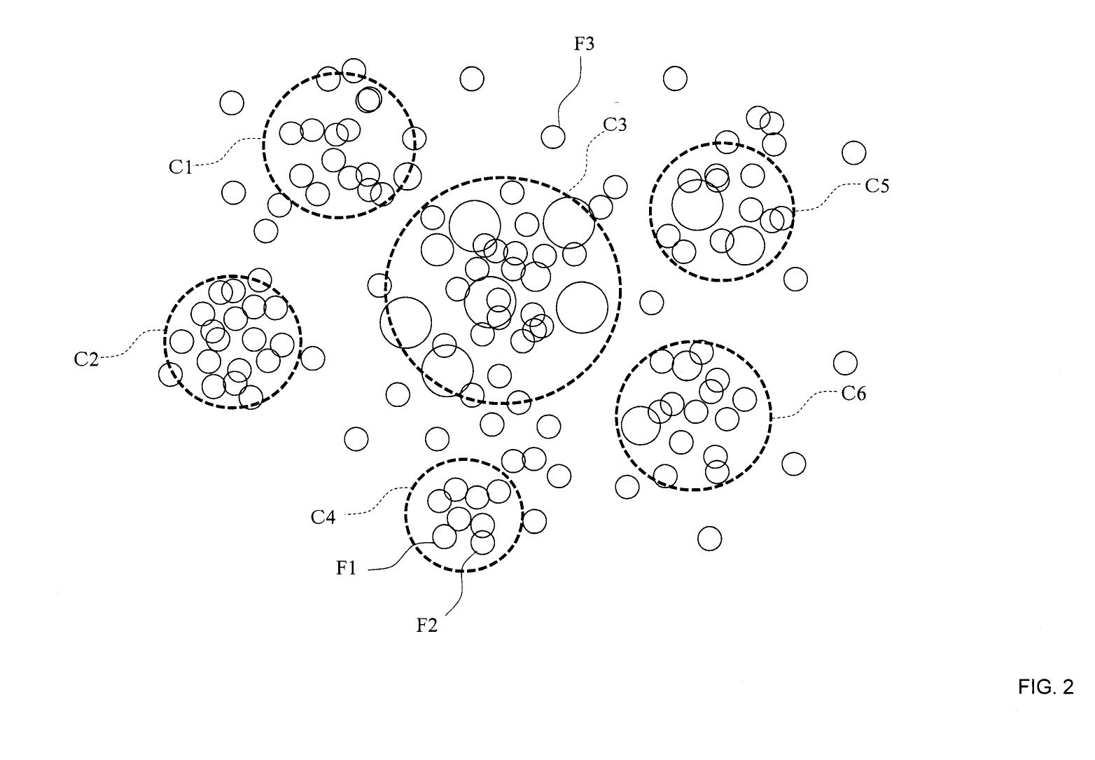

In the map creating procedure, a map creating unit 324 plots the segments on a plane based on the indexes. Specifically, the segments can be two-dimensionally plotted based on a mapping algorithm of “making close segments disposed nearly, and making far segments disposed away”. Hereinafter, such a map is called a “segment map”. The plotting plane may be a plane, or may be a curved surface such as a spherical surface. FIG. 2 illustrates an example of the map.

Since the segment data having similar spectrums is disposed within a short distance so as to form an area where segments are densely distributed, it is possible to sort out a segment group having a high similarity visually or using a certain algorithm. In a case where there is no need of visualization but only for the purpose of extracting the segments having a high similarity, a clustering method may be employed.

On the map, there generally appears an area having a high density or an area having a low density. The generation factors of these areas are different from each area. In the invention according to this embodiment, the generation factors of each area can be estimated by analyzing a characteristic frequency or the frequency spectrum of each area. Therefore, the invention according to this embodiment can make it possible to analyze big data which is generally considered to be difficult for the analysis.

### Second Embodiment

FIG. 1 illustrates an example of the data analysis apparatus according to this embodiment. In the data analysis apparatus according to this embodiment, the information processing unit 32 includes a division unit 321. In addition, in the data analysis method according to this embodiment, the information processing unit 32 performs a dividing procedure before the indexing procedure.

In the dividing procedure, the division unit 321 divides the numerical data into spatial or temporal sections so as to create segments, and stores the segments in a storage unit 31.

The data analysis apparatus 30 according to this embodiment can change the size of the segment or the attribute data according to the purpose of analysis, so that various analyses can be performed.

### Third Embodiment

Generally, there are a plurality of areas having a high density of segments, and each of the areas has a “characteristic spectrum”. In addition, there are frequency components which contributes to “closeness” of each segment data in each area, that is, the frequency component has small variance in amplitude for the segments in each area, and those frequency components will be referred to as a “characteristic frequencies” of each area.

FIG. 1 illustrates an example of the data analysis apparatus according to this embodiment. In the data analysis apparatus 30 according to this embodiment, the information processing unit 32 includes an area selecting unit 326 and a characteristic spectrum extracting unit 327. In addition, in the data analysis method according to this embodiment, the information processing unit 32 performs an area selecting procedure and a characteristic spectrum extracting procedure after the map creating procedure.

In the area selecting procedure, the area selecting unit 326 selects a certain area where the segments are distributed on the map. The certain area is, for example, areas C1 to C5 of which the density is equal to or more than a certain value. The certain area may be an area where the density is equal to or less than a certain value.

In the characteristic spectrum extracting procedure, the characteristic spectrum extracting unit 327 extracts the characteristic frequencies or the frequency spectrum in the area selected by the area selecting unit 326. For example, in a case where the area selecting unit 326 selects the area C1, the characteristic spectrum extracting unit 327 outputs the characteristic frequencies, the characteristic spectrum, or an average frequency spectrum of each segment contained in the area C1.

The numerical data is, for example, vibration data having a vibration frequency components of a vehicle. In this case, as will be described in fifth to eighth embodiments in detail, the certain area corresponds to a degraded state of the surface of a road where the vehicle runs, an abnormal state of the vehicle, or an abnormal state of driving.

The numerical data is, for example, vibration data having vibration frequency components of which at least a part of a biological body. In this case, as will be described in a ninth embodiment in detail, the certain area corresponds to a state of a part or all of the biological body. The vibration data is, for example, respiration, voice, electrocardiogram, echo, or a waveform of the movement of shoes or knees and so on.

The data analysis apparatus 30 according to this embodiment can analyze the characteristics of the segment data belonging to each area based on the characteristic spectrums or the characteristic frequencies, and can also analyze the generation factors thereof. In addition, an inherent tendency of overflowing data is visualized by plotting the corresponding data on the map with color using the attribute data of the segment as parameters, and various features are more easily discovered.

Furthermore, since the theory and the scheme of this embodiment are simple and can process big data on a framework at high speed, any person can analyze physical big data, and it is expected that there is no individual difference in the result.

### Fourth Embodiment

FIG. 1 illustrates an example of the data analysis apparatus according to this embodiment. In the data analysis apparatus 30 according to this embodiment, the information processing unit 32 includes a plotting unit 328. In addition, in the data analysis method according to this embodiment, the information processing unit 32 performs the indexing procedure and the plotting procedure after the map creating procedure.

There is a case where data is newly added according to the numerical data. In a case where the new numerical data is stored in the storage unit 31 and is analyzed, the indexing unit 323 performs the indexing procedure again. At this time, for the new segment, the indexing unit 323 calculates new indexes indicating similarity between the new segment and the existing segments already plotted on the map. The method of calculating the index is performed using the frequency spectrum similarly to the first embodiment.

In the plotting procedure, the plotting unit 328 plots the new segment in the vicinity of the segment similar to the new segment, based on the new indexes, among the plurality of existing segments on the map created by the map creating unit 324 for the existing data.

Since the new segment is plotted on the existing map, it is possible to ascertain whether the new segment is similar to the already existing segments. Therefore, it is possible to analyze the new segment with ease.

Hereinafter, the description will be made about an embodiment in which the numerical data is vibration data having the vibration frequency components of the vehicle.

### Fifth Embodiment

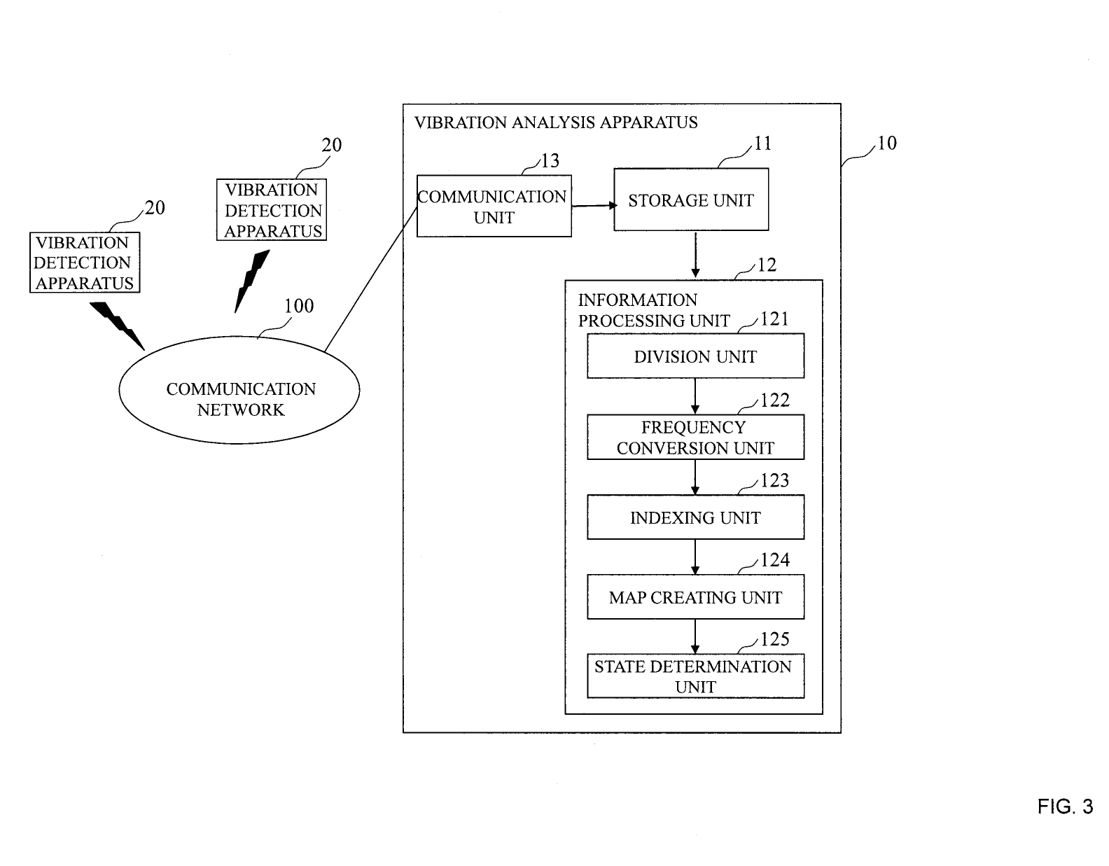

FIG. 3 illustrates a typical example of a vibration analysis system according to this embodiment. The vibration analysis system according to this embodiment includes a vibration analysis apparatus 10, a vibration detection apparatus 20, and a communication network 100. The vibration detection apparatus 20 is mounted on a vehicle, and detects a vibration. The communication network 100 transmits the vibration data detected by the vibration detection apparatus 20 to the vibration analysis apparatus 10.

The vehicle is, for example, an automobile which can run on a road or a train which can run on a railroad. The vibration detection apparatus 20 may be any device which can detect the vibration of the vehicle at least in a vertical direction (hereinafter, denoted as a z-axis direction), an advancing direction (hereinafter, denoted as an x-axis direction), or a direction (hereinafter, denoted as a y-axis direction) perpendicular to the vertical direction and the advancing direction. The vibration contains components having a slow vibration and a fast vibration, and is not limited in a frequency range.

The vibration may be detected using an acceleration sensor which can measure an acceleration applied to at least any one of the y-axis direction and the z-axis direction. The movement in the y-axis direction may be detected using a sensor which detects the rotation of a handle. In addition, the movement in the y-axis direction and the z-axis direction may be detected using an image or a moving image of the outside of the vehicle.

The vibration analysis apparatus 10 includes a storage unit 11, an information processing unit 12, and a communication unit 13. The communication unit 13 receives the vibration data detected by the vibration detection apparatus 20 from the communication network 100, and stores the vibration data in the storage unit 11. The information processing unit 12 reads the vibration data out of the storage unit 11, and performs a vibration analysis method according to this embodiment.

The vibration analysis method according to this embodiment includes the dividing procedure, a frequency converting procedure, the indexing procedure, the map creating procedure, and a state determining procedure in this order. The information processing unit 12 includes a division unit 121, a frequency conversion unit 122, an indexing unit 123, a map creating unit 124, and a state determination unit 125. The vibration analysis apparatus 10 may be realized by causing a computer to execute a vibration analysis program according to this embodiment. In this case, the respective configurations are realized by making the information processing unit 12 execute the vibration analysis program stored in the storage unit 11.

In the dividing procedure, the division unit 121 divides the vibration data of the vehicle into predetermined segments. For example, when the vibration data of segment i is set to Ai(t), the variation A(t) of the vibration data changes as follows.

A(t)=ΣAi(t),(i=1,2,3, . . . , N−1,N)

Herein, N is a total number of segments.

Herein, the segment corresponds to a geographical or temporal section, and is different according to the subject of analysis. For example, in a case where the state of a road or a railroad is analyzed, the segment becomes the geographical section, and in a case where the state of an operator is analyzed, the segment becomes the temporal section. The geographical section is, for example, a section in a unit of 100 m, and the temporal section is, for example, a section in a unit of 10 seconds.

In the frequency converting procedure, the frequency conversion unit 122 converts the vibration data Ai(t) of each segment i into a frequency spectrum Fi(ω).

In the indexing procedure, the indexing unit 123 calculates indexes indicating the similarity between the frequency spectrums Fi(ω) (i is arbitrary) of the respective segments. For example, the index indicating the similarity between segments i and k is denoted by Dᵢₖ.

For example, the frequency spectrum Fi(ω) is sampled at n discrete frequency values predetermined in advance. Then, Fi(ω) is converted into an n-dimensional vector Pi=(Fi(ω1), Fi(ω2), Fi(ω3), . . . , Fi(ωn−1), Fi(ωn)) using a frequency amplitude Fi(ωj) at a discrete frequency ωj. Therefore, the frequency spectrum of each segment can be converted into a vector. Herein, the number n for the sampling is arbitrary (for example, 100).

The indexing unit 123 calculates indexes indicating the similarity between the segments using the frequency spectrum Fi(ω). An index Dᵢₖ is an arbitrary index which can indicate the similarity of the frequency spectrum between the segments (for example, a distance between the vectors Pi and Pj). The index Dᵢₖ may be calculated using the inner product of the vectors Pi and Pj, or may be calculated using the outer product of the vectors Pi and Pj.

In the map creating procedure, the map creating unit 124 plots Pi (i=1, 2, 3, . . . , N−1, N) on the map based on the index Dᵢₖ. When the vectors are similar in the frequency spectrum, the vectors Pi and Pj are plotted close to each other on a map. As a basic algorithm, an algorithm similar to that disclosed in JP 2005-92442 A may be employed.

FIG. 2 illustrates an example of the map. The plot position on a plane corresponding to the n-dimensional vector Pi is set as Qi(xi, yi). In the plane, the segments similar in the frequency spectrum are plotted close to each other. For example, F1 and F2 similar in the frequency spectrum are closely disposed on the map, and F1 and F3 not similar in the frequency spectrum are disposed away from each other on the map.

In the state determining procedure, the state determination unit 125 distinguishes a cluster corresponding to an abnormality among clusters created by the segments on the map, and further determines a state and a level of the abnormality. In the case of a maintained road or railroad, the main cluster is considered to show that there is no abnormality. On the contrary, in a case where there is an abnormality, some small clusters other than the main cluster appear. The state determination unit 125 distinguishes a cluster corresponding to an abnormality of the vehicle or an abnormality of a route of the vehicle, and determines the presence or absence of the abnormality in each segment based on whether the segment is included in the subject cluster.

At this time, the segment already ascertained as having the abnormality is also plotted as a marker on the map, so that it is possible to determine that the cluster plotted with the marker is in the abnormal state similarly to the marker. For example, the frequency conversion unit 122 converts a marker segment into the frequency spectrum. Then, the state determination unit 125 distinguishes a cluster to which the marker segment belongs. Then, the state determination unit 125 determines that each segment included in the cluster to which the marker segment belongs has abnormality similar to the marker segment.

In the determining procedure, in a case where some of the segments forming the cluster are already ascertained to have an abnormal state, the other segments in the same cluster can be determined as being in the similar abnormal states. For example, in a case where some of the segments included in the cluster are ascertained as being abnormal, the state determination unit 125 determines that the respective segments included in the cluster to which the subject segments belongs are in the abnormal state.

In the determining procedure, in a case where the state of none of the segments in the cluster is ascertained, a field investigation is performed on some of the segments to determine the state thereof, so that the state of the remaining segments can be determined as being in the same state. For example, when an abnormal state of some segments included in the cluster is acquired, the state determination unit 125 determines that the respective segments included in the cluster to which the subject segment belongs are in the abnormal state similar to the subject segments.

In the determining procedure, the state determination unit 125 can also determine the abnormal states of the segments included in the other clusters C1, C2, and C4 to C6 using a difference between the frequency spectrum of the main cluster C3 containing the segments having no abnormality and the characteristic spectrums of the other clusters C1, C2, and C4 to C6 which do not belong to the subject main cluster.

As described above, the vibration analysis system, the vibration analysis apparatus, and the vibration analysis method according to this embodiment can detect the abnormality without using the information at a normal time.

When calculating the n-dimensional vector Pi, the indexing unit 123 may perform weighting on each frequency included in the vibration data. For example, the weighting value on the natural vibration frequency of the vehicle may be set to be small. Therefore, the cluster is formed clearly, so that the abnormality is easily detected.

The frequency spectrum may be changed depending on the speed of the vehicle. In order to cope with such a situation, a frequency correction unit (not illustrated) may be further included to correct a frequency change depending on the speed of the vehicle. The frequency correction unit (not illustrated) corrects that the frequency spectrum is shifted due to a change in the speed of the vehicle. In addition, the frequency correction unit (not illustrated) may exclude the data obtained at the time when the vehicle is stopped from the vibration data. In addition, the frequency correction unit (not illustrated) may extract only the vibration data obtained at the time when the speed of the vehicle is within a predetermined range speed. The division unit 121 divides the vibration data corrected by the frequency correction unit (not illustrated) into each segment.

### Sixth Embodiment

Herein, a specific example of this data analysis apparatus will be described.

The vibration analysis apparatus 10 according to this embodiment analyzes the state of a road on which the automobile runs. In this embodiment, the vibration detection apparatus 20 detects the vibration of the automobile in the vertical direction, and the segment is a geographical section of the road where the automobile runs.

Comparing the frequency spectrums of the respective segments, the segments of the road in the similar states have the similar frequency spectrums. In a case where the frequency spectrums are similar, the vectors Pi and Pj are plotted close to each other in the n-dimensional vector space.

On the map, the segments having the similar frequency spectrums are plotted close to each other, and form clusters. In the state determining procedure, the state determination unit 125 distinguishes an abnormal cluster corresponding to an abnormality of the road in each geographical section where the automobile runs among the clusters formed by the segments on the map and further determines the state and the level of the abnormality. As a result, it is possible to determine the state of the road in each geographical section where the automobile runs.

The main cluster is considered to be configured by segments in a good road state. A segment corresponding to a bridge or an overpass is considered as having relatively larger values in low frequency components, and considered to form a cluster different from the main cluster. Therefore, it is possible to analyze a degradation state of the road. In addition, the road having an underground cavity or a hollow portion is considered to form another cluster. Therefore, it is possible to analyze the state of the road. A segment in a large undulation also forms another cluster. In this way, various types of states such as the surface state or the structure of the road, the degradation, and the defect can be ascertained.

In the storage unit 11, as the numerical data, the vibration data measured on the road is stored. In this embodiment, all the vibration data from the beginning of acquiring the vibration data is stored. During that period, the road may be degraded or may be under pavement construction.

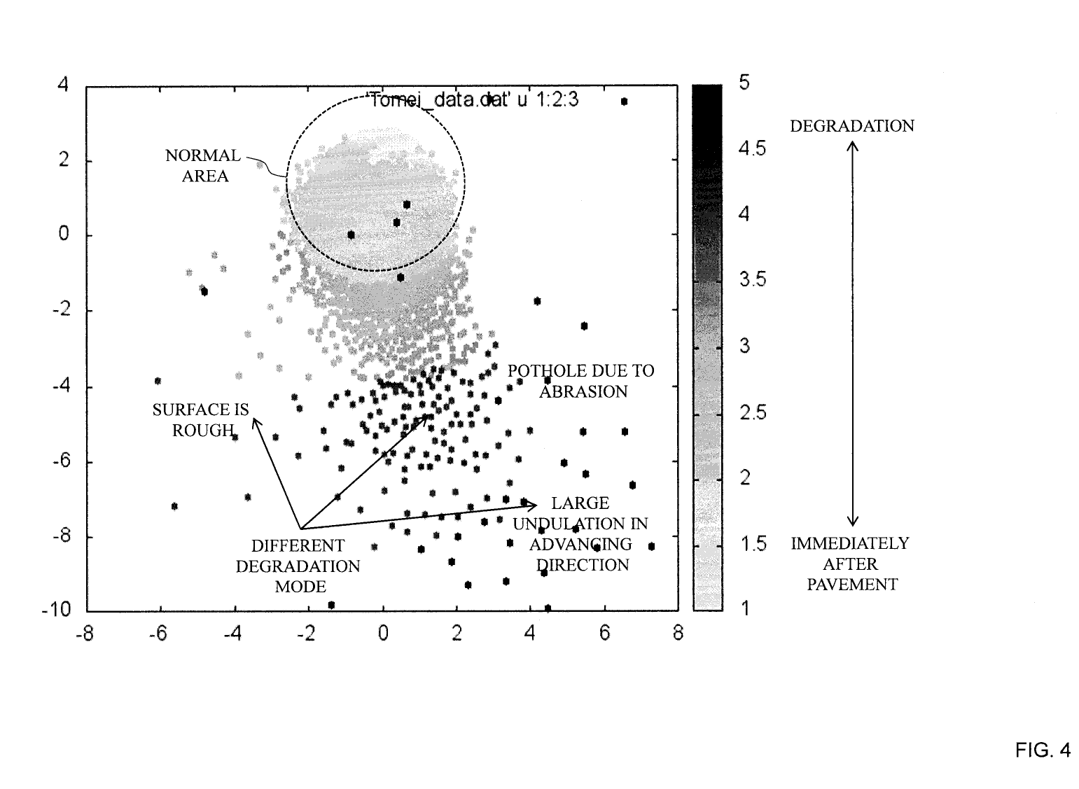

An example of the map of data acquired from an actual expressway is illustrated in FIG. 4. The difference in a shade of color 1 to 5 shows a magnitude of the vibration. A shade of color 1 portion and a shade of color 2 portion indicate segments having a small vibration, that is, a good surface state. On the other hand, an shade of color 4 portion and a shade of color 5 portion indicate segments having a large vibration, that is, a bad surface state.

In a well-managed road such as an expressway or a first-grade national road, a lot of segments are in a good surface state, the shade of color 1 portion and the shade of color 2 portion are plotted a lot, and form a large area (denoted as a normal area in the drawing). In a part of the segments, the degradation is progressed, and the plot position of the segment is gradually separated away from the normal area according to the progressing situation. The shade of color 3 area of FIG. 4 corresponds to the above case. Furthermore, when the degradation is progressed, the plot position of the segment is plotted with shade of color 4 or shade of color 5 as illustrated in FIG. 4. In this case, a degradation state (hereinafter, referred to as a degradation mode) is not uniform such that the road surface may be simply rough and a short-period vibration occupies a large portion, an intermediate-period vibration due to wheel tracks may mainly occur, and a long-period vibration due to an undulation in the advancing direction may occur, all of which cause different frequency spectrum patterns, so that the segments are distributed according to the degradation mode.

Therefore, the progressing state of the degradation and the degradation mode of each segment can be inversely determined from the plot position on the map.

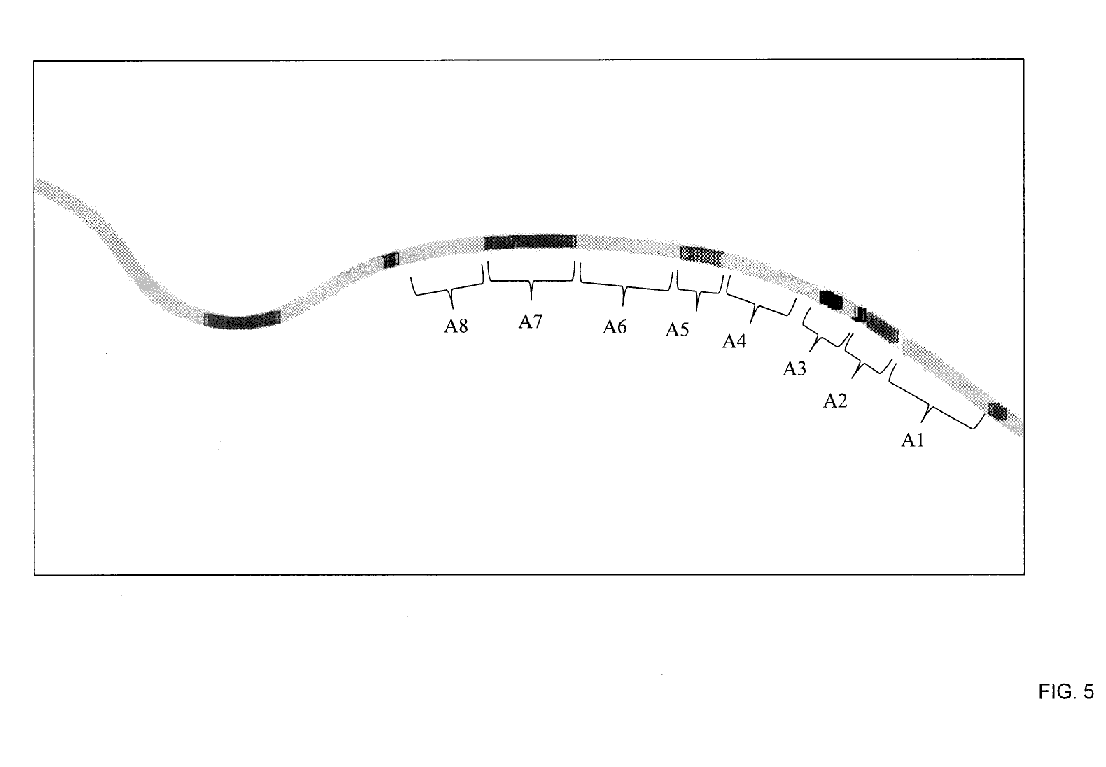

FIG. 5 illustrates an example of the degradation state of an actual road. A1 to A8 indicate sections of the road. Each section surrounded by a frame illustrated in the section indicates a segment. The sections A1, A4, A6, and A8 are sections where no degradation is progressed after the pavement. In the section A5, the degradation is progressed. The sections A2 and A7 are sections where the degradation is further progressed, and the section A3 is most progressed in degradation so that maintenance is necessary. The sections A2, A3, and A7 are estimated to be recovered after the maintenance into the good surface state similar to the section A1.

In the case of the road, the vibration data is added every day and accumulated. There are two methods of mapping the new data.

As a first method, for example, the everyday new data is added to the old data and the mapping is performed again using all the data. In this case, the indexes between the old data are not changed, but new indexes between the new data and the existing data and between the new data are necessarily calculated. On the created map, the plot positions of all the segments are slightly changed every day. In other words, the map corresponding to FIG. 4 is slightly changed every day.

As another method, there is a method of plotting the new data on a standard map which is a map created with the existing data. In this case, the indexes of the segments included in the new data and the segments of the old data are calculated, and the segments of the new data are plotted in the vicinity of the segment of the old data most similar to the new data. In this method, the shape of the standard map is not changed, and the daily new data is additionally plotted onto the map. In other words, in the map corresponding to FIG. 4, the existing plot position shows an image in which the data is newly plotted every day in general. Therefore, it can be said that this method is suitable for visualizing a daily change.

As described above, the vibration analysis system, the vibration analysis apparatus, and the vibration analysis method according to this embodiment can detect an abnormality of the road without using the information at a normal time. Therefore, the invention can sort out the segments to be maintained in priority. In addition, it is possible to analyze a progressing degree of the degradation of a bridge or an overpass.

### Seventh Embodiment

The vibration analysis apparatus 10 according to this embodiment analyzes an operation state of a driver who drives an automobile. In this embodiment, the vibration detection apparatus 20 detects the vibration of the automobile in the Y-axis direction, and the segment is a temporal section where the automobile runs.

Comparing the frequency spectrums of the respective segments, the frequency spectrum varies in a segment where the driving is swayed. In a case where the frequency spectrum is much different, the vectors Pi and Pj are plotted away from each other in the n-dimensional vector space.

On the map, the segments having the different frequency spectrums are plotted away from each other, and form clusters. In the state determining procedure, the state determination unit 125 distinguishes an abnormal cluster corresponding to an abnormality of the driving in each temporal section where the automobile runs among the clusters formed by the segments on the map and further determines the state and the level of the abnormality. As a result, it is possible to determine the state of the driver in each temporal section where the automobile runs.

A normal driving state forms the largest cluster. The “swaying” of the handle is represented as a difference of the frequency spectrum in the Y-axis. A swaying level of the driving is considered to vary according to a drowsy driving, a drunken driving, a driving in a fatigue state, a driving in a poor physical state, a rapid lane change, and the like, and the segments on the map form the corresponding clusters for the respective cause. In this way, various driving states form the different clusters.

Furthermore, in this embodiment, a biological information detecting apparatus (not illustrated) is preferably provided to detect biological information of the driver of the vehicle. The biological information may be any information through which the condition of body and mind of the driver can be estimated, for example, pulsation, amount of perspiration, a body temperature, a movement of the eyeball, a posture, and information derivable therefrom. The pulsation, the amount of perspiration, and the body temperature are detected, for example, using a sensor provided in the handle. The movement of the eyeball and the posture are detected, for example, by processing the captured image of the driver.

It is possible to estimate whether the driver becomes tense by detecting the change in the pulsation or the amount of perspiration. In addition, it is possible to estimate whether the driver is drowsy by detecting a reduction of the body temperature. In addition, it is possible to estimate whether attentiveness of the driver is lowered by detecting a reduction of the movement of the eyeball. In addition, it is possible to estimate whether the driver is drowsy by detecting a forward-bent posture of the head.

The biological information detected by the biological information detecting apparatus (not illustrated) is divided into each segment which is the temporal section similarly to the vibration data, and added to the frequency spectrum Fi(ω) to determine the similarity. For example, in a case where the frequency spectrum Fi(ω) of the vibration data is an n-dimensional space, the biological information is added to the (n+1)-dimensional space and the subsequent spaces. In this way, it is possible to calculate an index indicating the similarity between the segments by adding the biological information dimension to the frequency spectrum Fi(ω).

Further, in a case where there are a plurality of biological information, the number of dimensions of the frequency spectrum Fi(ω) may be increased. Regarding the number of dimensions used in the biological information, one type of the biological information may be assigned to one dimension, or may be assigned to two or more dimensions. The dimension used for the biological information may be weighted according to a degree of contribution of the biological information.

As described above, the vibration analysis system, the vibration analysis apparatus, and the vibration analysis method according to this embodiment can detect an abnormality of the driving without using the information at a normal time.

### Eighth Embodiment

The vibration analysis apparatus 10 according to this embodiment analyzes the state of a railroad on which a train runs. In this embodiment, the vibration data is data obtained by detecting a vibration of the train in the Z-axis direction and the Y-axis direction, and the segment is a geographical section where the train runs.

In this embodiment, temporal changes in the vibration data in the Z-axis direction and the Y-axis direction are Az(t) and Ay(t), respectively. In the frequency converting procedure, the frequency conversion unit 122 converts the vibration data in the Z-axis direction and the Y-axis direction into the frequency spectrums.

The following processes are basically performed by combining the data in the Y-axis and the data in the Z-axis, but may handle the data individually for the purpose of the analysis. The processes of the indexing procedure and the map creating procedure are the same as those in the first and the second embodiments.

Comparing the frequency spectrums of the respective segments, the segments in the similar railroad states should have the similar frequency spectrums. In a case where the frequency spectrums are similar, the vectors Pi and Pj are plotted close to each other in the n-dimensional vector space.

On the map, the segments having the similar frequency spectrums are plotted close to each other, and form clusters. In the state determining procedure, the state determination unit 125 distinguishes an abnormal cluster corresponding to an abnormality of the railroad in each geographical section where the train runs among the clusters formed by the segments on the map and determines the state and the level of the abnormality. As a result, it is possible to determine the state of the railroad in each geographical section where the train runs.

The segments of a normal state form the largest cluster. The vertical undulation of the railroad is reflected on the frequency spectrum in the Z-axis direction. A fluctuation of the width between rails influences the frequency spectrums in the Z-axis direction and the Y-axis direction. In this way, since various states form the different clusters, it is possible to analyze various states such as the degradation and the defect of the railroad. In addition, it is possible to analyze the state of the vehicle such as the degradation, the defect, and the actual loadage of the vehicle.

As described above, the vibration analysis system, the vibration analysis apparatus, and the vibration analysis method according to this embodiment can detect an abnormality of the railroad without using the information at a normal time.

### Ninth Embodiment

Hereinafter, an embodiment in a case in which the numerical data is vibration data having vibration frequency components of which at least a part of a biological body will be described.

### (A) Analysis on Respiration

A certain variation (for example, a respiratory sound or a physical variation of a part of body) of the body according to the respiration is divided in a unit of a certain time (for example, a unit of 20 seconds; hereinafter, referred to as a segment), and the variation in the segment is converted into the frequency spectrum. Enormous volume of respiration data in sleeping state obtained from healthy people and patients is divided into the segments, and the map can be created based on the similarity of the frequency spectrums. Since various respiration states containing normal and abnormal conditions are associated with the respective areas of the map, it is possible to inversely determine the state of the respiration by confirming a position plotted on the map. Using this map as the “standard map”, when the respiration data of a person as a diagnoses target is divided into segments, and the respective segments are plotted in the vicinity of the similar segments on the standard map, the segments of the respiration of a person can be plotted on the standard map. The soundness of the respiration of a person can be evaluated based on this distribution, or in a case where there is a problem, the cause can be estimated.

### (B) Analysis on Electrocardiogram

An electrocardiogram is divided into a unit of a certain time (for example, a unit of 1 minutes; hereinafter, referred to as a segment), and the variation in the segment is converted into the frequency spectrum. Therefore, not only the possibility to determine “Abnormality of Heart” of a person but also an abnormal pattern thereof can be determined by plotting the segments of the patient on the standard map.

### (C) Analysis on Other Internal Organs

It is possible to distinguish “Abnormality” based on a result of measuring the movement of esophagus, stomach, intestine, and lung so as to estimate the cause of the abnormality. As a specific measurement method, there is a method of measuring a variation of a voltage using a piezoelectric element, or a method of measuring a movement through a moving image using an echo.

### (D) Analysis on Walking

Under a constant walking condition, a waveform of the movement of shoes or knees at the time of walking is measured. The measured waveform is divided into a unit of a certain time (for example, a unit of 1 minute; hereinafter, referred to as a segment), and the variation in the segment is converted into the frequency spectrum. Similarity between many segments obtained from the walking result of many subject people can be represented as indexes from the frequency spectrums.

A walking tendency is generated from various physical and psychological causes, and the characteristic of the walking is considered to be different in each cause. The standard map is created from the segment data of many patients. In the standard map, the areas are divided according to a pattern of the walking. When the segments of a new patient are plotted on the standard map, it is possible to estimate the physical and psychological causes of the walking tendency of the patient according to the area where the segment data is plotted on the standard map.

As a result, the “walking abnormality” of the person can be determined, and the cause of the walking abnormality can be estimated by plotting the segment data of the patient on the standard map.

### (E) Analysis on Voice

In a call center and so on, there may often cause trouble such that a customer feels aggrieved at an operator's care. For this reason, if it is possible to grasp the customer's emotion at any time, the trouble can be avoided from being serious by quickly changing the operator in charge to an experienced operator or a chief manager as needed. In many cases, it is not possible to grasp the customer's emotion exactly only by the wording, so that the method of grasping the emotion from the voice is effective.

Specifically, many accumulated samples of customer's voices are divided into segments in a certain time, and a map is created based on a similarity between the frequency spectrums of the respective segments. The cases when the customers became emotional are recorded as attribute data, a heat map can be plotted by classifying colors (for example, blue to red) in an ascending order of emotion according to the density of segments on the map indicating the case where the customers felt aggrieved, and the heat map is set to the “standard map”. Since the nature of voice is different from person to person, it cannot be said that the red area is limited to only one portion. In the case of a phone call from a new customer, when sequentially plotting the segment on the standard map, the emotion is generally plotted in a blue area when the customer is emotionally stable, but since the plot position is gradually shifted to the red area when the emotion becomes tensed, a danger can be sensed based on the plot position and the operator can be replaced with an operator who can appropriately handle that situation, so that the trouble can be avoided in advance.

### (Note)

In a vibration analysis apparatus and a vibration analysis method according to the note, when each geographical or temporal section is plotted on a plane based on indexes indicating similarity between the frequency spectrums in each section, a cluster is formed by plotted sections in similar according to the state of the section. Since the sections are automatically classified into clusters having the similar states, it is possible to determine a section having a specific state in addition to an abnormal section.

Specifically, the vibration analysis apparatus according to the note includes

a division unit configured to divide vibration data of a vehicle into each segment which is a predetermined geographical or temporal section,

a frequency conversion unit configured to convert the vibration data into a frequency spectrum in each segment,

an indexing unit configured to calculate an indexes indicating similarity between the segments using similarity between the frequency spectrums of the segments,

a map creating unit configured to plot the segment on a map based on the indexes, and

a state determination unit configured to distinguish a cluster corresponding to an abnormality of the vehicle or an abnormality of a route of the vehicle among clusters containing the segments on the map, and determine the presence or absence of the abnormality in each segment based on whether the segment is included in the subject cluster.

The vibration analysis apparatus according to the note may be configured such that the frequency conversion unit converts the segment ascertained as already having an abnormal state into the frequency spectrum as a marker segment, and the state determination unit distinguishes a cluster to which the marker segment belongs and determines whether there is the same abnormality as the marker segment in each segment contained in the subject cluster.

The vibration analysis apparatus according to the note may be configured such that in a case where it is determined that a part of the segments in the cluster have abnormalities, the state determination unit determines that each segment contained in the cluster to which the subject segments belongs is in the abnormal state.

The vibration analysis apparatus according to the note may be configured such that when the abnormality contents of a part of the segments contained in the cluster is acquired, the state determination unit determines that each segment contained in the cluster to which the subject segments belong is in the abnormal state of the abnormality contents.

The vibration analysis apparatus according to the note may be configured such that the state determination unit determines the abnormal state of the segment contained in another cluster using a difference between the frequency spectrum of a main cluster formed by the segments having no abnormality and the frequency spectrum of the another cluster not contained in the main cluster.

The vibration analysis apparatus according to the note may be configured such that the vibration data is data obtained by detecting a movement of an automobile in the vertical direction, the segment is a geographical section of a road where the automobile runs, and the state determination unit distinguishes a cluster corresponding to an abnormality of the road in each geographical section where the automobile runs among the clusters formed by the segments on the map.

The vibration analysis apparatus according to the note may be configured such that the vibration data is data obtained by detecting a vibration of the automobile in the direction perpendicular to the vertical and advancing directions of the vehicle, the segment is a temporal section where the automobile runs, and the state determination unit distinguishes a cluster corresponding to an abnormality of a driver in each temporal section where the automobile runs among the clusters formed by the segments on the map.

The vibration analysis apparatus according to the note may be configured such that the vibration data is data obtained by detecting a movement of a train in the advancing direction of the vehicle, a direction perpendicular to the vertical direction, and the vertical direction, the segment is a geographical section of a railroad where the train runs, and the state determination unit distinguishes a cluster corresponding to an abnormality of the railroad in each geographical section where the train runs among the clusters formed by the segments on the map.

Specifically, a vibration analysis system according to the note includes a vibration detection apparatus configured to be mounted on a vehicle and to detect a vibration, a communication network configured to transmit vibration data detected by the vibration detection apparatus, and a vibration analysis apparatus configured to determine whether there is the abnormality of the vehicle or the railroad of the vehicle using the vibration data transmitted through the communication network.

Specifically, the vibration analysis method according to the note includes a dividing procedure configured to divide vibration data of a vehicle into each segment which is a predetermined geographical or temporal section, a frequency converting procedure configured to convert the vibration data into the frequency spectrum in each segment, an indexing procedure configured to calculate an indexes indicating similarity between the segments using similarity between the frequency spectrums of the segments, a map creating procedure configured to plot the segments on a map based on the indexes, and a state determining procedure configured to distinguish a cluster corresponding to an abnormality of the vehicle or an abnormality of a path of the vehicle among the clusters formed by the segments on the map, and determine the presence or absence of the abnormality in each segment based on whether the segment is included in the subject cluster.

Specifically, a vibration analysis program according to the note causes a computer to execute a dividing procedure in which a division unit divides vibration data of a vehicle into each segment which is a predetermined geographical or temporal section, a frequency converting procedure in which a frequency conversion unit converts the vibration data into a frequency spectrum for each segment, an indexing procedure in which an indexing unit calculates indexes indicating similarity between the segments using similarity between the frequency spectrums of the segments, a map creating procedure in which a map creating unit plots the segment on a map based on these indexes, and a state determining procedure in which a state determination unit distinguishes a cluster corresponding to an abnormality of the vehicle or an abnormality of a path of the vehicle among the clusters formed by the segments on the map, and determines the presence or absence of the abnormality in each segment based on whether the abnormality is included in the cluster.

According to the invention described in the note, since the index indicating the similarity between the frequency spectrums of the vibration data of each segment is used, it is possible to detect the abnormal segment without using information at a normal time and to determine the state of the abnormal state. Therefore, according to the invention described in the note, it is possible to provide a vibration analysis method through which the abnormal segment can be detected without using information at a normal time and the state of the abnormal state can be determined.

### INDUSTRIAL APPLICABILITY

The invention can be applied to a transportation infrastructure business, a transportation service business, a vehicle management business, and a construction business.

### REFERENCE SIGNS LIST

10: vibration analysis apparatus
11, 31: storage unit
12, 32: information processing unit
13: communication unit
20: vibration detection apparatus
121, 321: division unit
122: frequency conversion unit
123, 323: indexing unit
124, 324: map creating unit
125: state determination unit
30: data analysis apparatus
326: area selecting unit
327: characteristic spectrum extracting unit
328: plotting unit

1. A data analysis apparatus comprising:
an indexing unit configured to calculate indexes each indicating similarity between each pair in a plurality of segments, each of which is numerical data having at least one frequency component, using a frequency spectrum containing the frequency component of the segment; and
a map creating unit configured to plot the segments on a plane based on the indexes.

2. The data analysis apparatus according to claim 1, further comprising
an area selecting unit configured to select a certain area in which the segments are plotted on the plane.

3. The data analysis apparatus according to claim 2, further comprising
a characteristic spectrum extracting unit configured to extract a characteristic frequency or the frequency spectrum of all segments in the area selected by the area selecting unit.

4. The data analysis apparatus according to claim 2,
wherein the numerical data contains vibration frequency components of a vehicle, and
wherein the certain area corresponds to a degradation or a state of the vehicle or a path where the vehicle runs, or a driving state of the vehicle.

5. The data analysis apparatus according to claim 3,
wherein the numerical data contains vibration frequency components of a vehicle, and
wherein the certain area corresponds to a degradation or a state of the vehicle or a path where the vehicle runs, or a driving state of the vehicle.

6. The data analysis apparatus according to claim 2,
wherein the numerical data includes vibration frequency components of which at least a part of a biological body, and
wherein the certain area corresponds to a state of the part or all of the biological body.

7. The data analysis apparatus according to claim 3,
wherein the numerical data includes vibration frequency components of which at least a part of a biological body, and
wherein the certain area corresponds to a state of the part or all of the biological body.

8. The data analysis apparatus according to claim 1,
wherein the indexing unit calculates new indexes indicating similarity between a new segment different from the plurality of segments and the plurality of segments using the frequency spectrum of the new segments and spectrums of the plurality of segments,
the data analysis apparatus further comprising a plotting unit configured to plot the new segment in the vicinity of a segment similar to the new segment among the plurality of segments based on the new indexes.

9. The data analysis apparatus according to claim 1, further comprising
a division unit configured to divide the numerical data into spatial or temporal sections to create the segments.

10. The data analysis apparatus according to claim 2, further comprising
a division unit configured to divide the numerical data into spatial or temporal sections to create the segments.

11. The data analysis apparatus according to claim 3, further comprising
a division unit configured to divide the numerical data into spatial or temporal sections to create the segments.

12. The data analysis apparatus according to claim 4, further comprising
a division unit configured to divide the numerical data into spatial or temporal sections to create the segments.

13. The data analysis apparatus according to claim 5, further comprising
a division unit configured to divide the numerical data into spatial or temporal sections to create the segments.

14. The data analysis apparatus according to claim 6, further comprising
a division unit configured to divide the numerical data into spatial or temporal sections to create the segments.

15. The data analysis apparatus according to claim 7, further comprising
a division unit configured to divide the numerical data into spatial or temporal sections to create the segments.

16. The data analysis apparatus according to claim 8, further comprising
a division unit configured to divide the numerical data into spatial or temporal sections to create the segments.

17. A data analysis method which is performed by a data analysis apparatus, comprising:
an indexing procedure in which the data analysis apparatus calculates indexes indicating similarity between a plurality of segments, each of which is numerical data having at least one frequency component, using a frequency spectrum containing the frequency component of the segment; and
a map creating procedure in which the data analysis apparatus plots the segments on a plane based on the indexes.
